(19) United States
(12) Patent Application Publication
(10) Pub. No.: US 2020/0368005 A1
(43) Pub. Date: Nov. 26, 2020

(54) POLYELECTROLYTE DENTAL COATINGS

(71) Applicant: SAFEWHITE, INC., Columbus, OH (US)

(72) Inventors: Satish Kumar Garre Venkata Raghavendra, Dublin, OH (US); Michelle Anne Hurtubise, Kettering, OH (US); Carlos Eduardo Martinez, Cincinnati, OH (US); Deepti Gupta, Centerville, OH (US); Roopa Comandur, Columbus, OH (US); Brandon Thomas Moore, Houston, TX (US)

(21) Appl. No.: 16/651,980

(22) PCT Filed: Oct. 1, 2018
(86) PCT No.: PCT/US18/53764

§ 371 Date: Mar. 27, 2020

Related U.S. Application Data
(60) Provisional application No. 62/565,275, filed on Sep. 29, 2017.

(51) Int. Cl. A61C 19/06; A61K 8/29; A61K 8/28; A61K 8/27; A61K 8/26; A61K 8/19; A61Q 11/00; A61Q 19/02; A61Q 5/08; A61Q 3/02

(52) U.S. Cl. CPC A61C 19/066; A61K 8/29; A61K 8/28; A61K 8/27; A61Q 3/02; A61K 8/19; A61Q 11/00; A61Q 19/02; A61Q 5/08; A61K 8/26

(57) ABSTRACT

This document relates to materials and methods involved in providing a substrate (e.g., tooth, tooth component, inorganic dental material, skin, hair, nails, hair, lips, and eyelashes) with a desired benefit (e.g., appearance, flavor, breath freshening, and/or medicament). For example, materials and methods for contacting a tooth, tooth component, or inorganic dental material with a polyelectrolyte adhesive containing a conjugate of a polyamine (e.g., a naturally-derived polyamine and/or a synthetic polyamine), a polyphosphate, and one or more whitening agents to provide the substrate with a change in appearance (e.g., a whiter appearance, a more natural appearance, and/or a glossy appearance) are provided.

### CROSS-REFERENCE TO RELATED APPLICATIONS

This application is a National Stage Application under 35 U.S.C. § 371 and claims the benefit of International Application No. PCT/US2018/053764, filed Oct. 1, 2018, which claims priority to U.S. Application No. 62/565,275, filed Sep. 29, 2017, entitled POLYELECTROLYTE DENTAL COATINGS, the disclosure of which is incorporated herein by reference.

### BACKGROUND

### 1. Technical Field

This document relates to materials and methods involved in providing a substrate (e.g., tooth, tooth component, inorganic dental material, skin, hair, nails, hair, lips, and eyelashes) with a desired benefit (e.g., appearance, flavor, breath freshening, and/or medicament). For example, this document relates to methods and materials for contacting a tooth, tooth component, or inorganic dental material with a polyelectrolyte adhesive containing a conjugate of a polyamine (e.g., a naturally-derived polyamine and/or a synthetic polyamine), a polyphosphate, and one or more whitening agents to provide the substrate with a change in appearance (e.g., a whiter appearance, a more natural appearance, and/or a glossy appearance).

### 2. Background Information

In general, white teeth are considered cosmetically desirable as they make a person feel younger and confident. Lack of personal oral hygiene and care among others can lead to teeth discoloration. The tooth structure that is generally responsible for presenting a stained appearance is the enamel layer. Various factors contribute to natural teeth discoloration, since they are exposed to various foods and coloring pigments both natural and artificial. For example, the formation of plaque and tartar matrices on the tooth surface can entrap stains, thereby leading to enamel discoloration.

Many consumer tooth whitening preparations have been developed to address the cosmetic preference and to restore luster to tooth enamel discolored by surface entrapped materials and stains. While all dentifrices and mouthwashes contain some cleaning, bleaching and polishing agents, some enamel deposits become intractable to being fully removed by these agents under normal use conditions. Tobacco users often develop discolored enamel because the tars and particulates in tobacco collect on the teeth. In some cases, foods and drinks (e.g., tea) and/or antibiotics (e.g., tetracycline) can stain or discolor tooth enamel.

Consumer products have also been developed to lighten skin color. Such products are often used to match areas of abnormally high pigmentation (e.g., lentigo spots, moles and birthmarks) to the surrounding skin. In addition, temporary alteration of the skin color is also a desired change, and achieved with different means. Providing the skin and other organic substrates with a longer lasting change in appearance is a universally desired benefit

### SUMMARY

This document provides materials and methods for contacting a tooth, tooth component, or inorganic dental material (e.g., a human tooth, a human tooth component, or an inorganic dental material within a human's mouth) with a polyelectrolyte adhesive containing a conjugate of at least a polyamine (e.g., a naturally-derived polyamine and/or a synthetic polyamine), a polyphosphate, and one or more beneficial agents (e.g., one or more whitening agents) to provide the tooth, tooth component, or inorganic dental material with a desired benefit (e.g., whiter appearance, more natural appearance, glossy appearance, flavor, breath freshening, and/or medicament). The materials and methods provided herein can allow a person to easily apply (e.g., self-apply) a composition (e.g., a liquid composition) containing a polyelectrolyte adhesive. For example, a polyelectrolyte adhesive provided herein can adhere or attach a beneficial agent to teeth as a coating to provide a desired benefit to the coated teeth. In some cases, a coating can be resistant to abrasion (e.g., mechanical abrasion and/or bio-mechanical forces). In some cases, teeth having a desired benefit can be obtained using the methods and materials provided herein without harsh bleaching or de-staining techniques.

As described herein, a polyelectrolyte adhesive containing a conjugate of at least a polyamine, a polyphosphate, and a beneficial agent (e.g., a whitening agent such as TiO₂) can be applied to teeth using a brush to achieve a uniform and easy-to-apply coating with no lasting brush marks or other implement markings. For example, a polyelectrolyte adhesive containing a naturally-derived polyamine, a polyphosphate, and a whitening agent (e.g., TiO₂) can be used to provide teeth with a whiter appearance. For example, a polyelectrolyte adhesive containing a conjugate of at least a naturally-derived polyamine, a polyphosphate, and a flavor (e.g., mint oil) can be used to provide a polyelectrolyte adhesive coating with a pleasant flavor and provide teeth with fresher breath.

In one aspect, this document provides polyelectrolyte adhesives. In some embodiments, a polyelectrolyte adhesive can include a conjugate of a polyamine, a polyphosphate, and a whitening agent and can have the ability to interact with or bind to a substrate under wet conditions (e.g., saliva), where the whitening agent of provides the substrate with a white appearance. The substrate can be a tooth, a tooth component, an inorganic dental material, skin, hair, or nails. When the substrate is a tooth, the tooth can be a human tooth. The polyamine can include a synthetic polyamine selected from polyallylamine (PAA), polydiallylamine (DAA), polyampholite, polylysine, polyvinyl amine, polyethyleneimine (PEI), heparin, polymethacrylate (e.g., EUDRAGIT® E 100, EUDRAGIT® EPO, EUDRAGIT® RL30D, EUDRAGIT® RS30D, EUDRAGIT® L30D-55), and polyhistidine. The polyamine can include a naturally-derived polyamine selected from deacetylated chitosan, soy-based polyamines, spermine, spermidine, putrescine, polyamines extracted from wheat germ, and polyamines extracted from rice.

The polyphosphate can be sodium hexametaphosphate, sodium trimetaphosphate, sodium tripolyphosphate, sodium pyrophosphate, potassium tripolyphosphate, potassium pyrophosphate, or adenosine triphosphate. The polyelectrolyte adhesive can have a w/w ratio of polyamine to polyphosphate of about 20:1 to about 1:20 (e.g., about 10:1, about 5:1, about 3:1, about 1:1, about 1:3, about 1:5, about 1:6.7, or about 1:10). The whitening agent can include at least one of titanium dioxide, hydroxyapatite, silicon dioxide, zinc oxide, aluminum oxide, zirconium oxide, sodium silicate, alumino silicate, and bioactive glass. When the whitening agent includes TiO₂, the TiO₂ can be from about 0.005% to about 10% of the polyelectrolyte adhesive. The polyelectrolyte adhesive also can include a rigidity promoter (e.g., chitosan, zinc oxide, zirconium oxide, aluminum oxide, and silicates). When the rigidity promoter include ZnO, the ZnO can be from about 0.05% to about 50% of the polyelectrolyte adhesive. The polyelectrolyte adhesive also can include an opacifier (e.g., apatite, hydroxyapatite, mica, titanated mica, zinc oxide, zirconium oxide, aluminum oxide, boron trioxide, bioactive glass, sodium oxide, sodium silicate, aluminum silicate, and zirconium silicate). In some cases, when the opacifier is bioactive glass sodium silicate, or aluminum silicate, and the polyelectrolyte adhesive is applied under wet conditions, the polyelectrolyte adhesive can form a clear coating. In some cases, when the opacifier is bioactive glass sodium silicate, or aluminum silicate, and the polyelectrolyte adhesive is applied under dry conditions, the polyelectrolyte adhesive can form a white coating. The polyelectrolyte adhesive also can include a stain resistance agent (e.g., silane coated oxides, Carbopol® polymers, magnesium stearate, silanes, silicates, hydrophobic lipids, and amphiphilic lipids). The polyelectrolyte adhesive also can include a flavoring agent (e.g., spearmint oil, peppermint oil, wintergreen oil, vanilla, citrus oil, and cinnamon oil). The polyelectrolyte adhesive also can include a coolant (e.g., menthol or a carboxamide cooling agent).

In another aspect, this document provides a method for altering an appearance of a substrate. This method can include, or consist essentially of, applying to a substrate a polyamine, a polyphosphate, and a beneficial agent, to form a polyelectrolyte adhesive on a surface of the substrate, where the beneficial agent alters the appearance of the substrate. The substrate can be a tooth, a tooth component, an inorganic dental material, skin, hair, and nails. When the substrate is a tooth, the tooth can be a human tooth. The beneficial agent can be a whitening agent, and the method can include altering the appearance of the teeth such that said substrate appears whiter. The whitening agent can include at least one of titanium dioxide, hydroxyapatite, silicon dioxide, zinc oxide, aluminum oxide, zirconium oxide, sodium silicate, alumino silicate, and bioactive glass. When the whitening agent includes TiO₂, the TiO₂ can be about 0.005% to about 10% of the polyelectrolyte adhesive. The beneficial agent can be an opacifier, and the method can include altering the appearance of the substrate such that the substrate appears more white, more natural looking, and/or more glossy looking. The opacifier can be apatite, hydroxyapatite, mica, titanated mica, zinc oxide, zirconium oxide, aluminum oxide, boron trioxide, bioactive glass, sodium oxide, sodium silicate, aluminum silicate, or zirconium silicate. The polyamine can include a synthetic polyamine selected from the group consisting of PAA, DAA, polyampholite, polylysine, polyvinyl amine, PEI, heparin, polymethacrylate (e.g., EUDRAGIT® E 100, EUDRAGIT® EPO, EUDRAGIT® RL30D, EUDRAGIT® RS30D, EUDRAGIT® L30D-55), and polyhistidine. The polyamine can include a naturally-derived polyamine selected from the group consisting of deacetylated chitosan, soy-based polyamines, spermine, spermidine, putrescine, polyamines extracted from wheat germ, and polyamines extracted from rice. The polyphosphate can be sodium hexametaphosphate, sodium trimetaphosphate, sodium tripolyphosphate, sodium pyrophosphate, potassium tripolyphosphate, potassium pyrophosphate, or adenosine triphosphate. The polyelectrolyte adhesive can include a w/w ratio of polyamine to polyphosphate of about 20:1 to about 1:20 (e.g., about 10:1, about 5:1, about 3:1, about 1:1, about 1:3, about 1:5, about 1:6.7, or about 1:10). The applying step can occur under wet conditions (e.g., in the presence of saliva).

In another aspect, this document provides an applicator strip including a polyamine, a polyphosphate, and a beneficial agent. The polyamine, polyphosphate, and whitening agent can form a polyelectrolyte adhesive on a surface of a substrate when said applicator strip is applied to the substrate, where the beneficial agent of the polyelectrolyte adhesive alters the appearance of the substrate, and where the polyelectrolyte adhesive can adhere to the substrate under wet conditions (e.g., in the presence of saliva). The substrate can be a tooth, a tooth component, an inorganic dental material, skin, hair, or nails. When the substrate is a tooth, the tooth can be a human tooth. The applicator polyamine can include a synthetic polyamine selected from the group consisting of PAA, DAA, polyampholite, polylysine, polyvinyl amine, PEI, heparin, polymethacrylate (e.g., EUDRAGIT® E 100, EUDRAGIT® EPO, EUDRAGIT® RL30D, EUDRAGIT® RS30D, EUDRAGIT® L30D-55), and polyhistidine. The polyamine can include a naturally-derived polyamine selected from the group consisting of deacetylated chitosan, soy-based polyamines, spermine, spermidine, putrescine, polyamines extracted from wheat germ, and polyamines extracted from rice. The polyphosphate can be sodium hexametaphosphate, sodium trimetaphosphate, sodium tripolyphosphate, sodium pyrophosphate, potassium tripolyphosphate, potassium pyrophosphate, or adenosine triphosphate. The applicator strip can be a dissolvable strip. The applicator strip can include a detachable backing.

In another aspect, this document provides a powder including a polyamine, a polyphosphate, and a beneficial agent. The polyamine, polyphosphate, and beneficial agent can form a polyelectrolyte adhesive on a surface of a substrate when the powder is applied to the substrate, where the beneficial agent of the polyelectrolyte adhesive alters the appearance of the substrate, and where the polyelectrolyte adhesive can adhere to the substrate under wet conditions (e.g., in the presence of saliva). The substrate can be a tooth, a tooth component, an inorganic dental material, skin, hair, and nails. When the substrate is a tooth, the tooth can be a human tooth. The polyamine can include a synthetic polyamine selected from the group consisting of PAA, DAA, polyampholite, polylysine, polyvinyl amine, PEI, heparin, polymethacrylate (e.g., EUDRAGIT® E 100, EUDRAGIT® EPO, EUDRAGIT® RL30D, EUDRAGIT® RS30D, EUDRAGIT® L30D-55), and polyhistidine. The polyamine can include a naturally-derived polyamine selected from the group consisting of deacetylated chitosan, soy-based polyamines, spermine, spermidine, putrescine, polyamines extracted from wheat germ, and polyamines extracted from rice. The polyphosphate can be sodium hexametaphosphate, sodium trimetaphosphate, sodium tripolyphosphate, sodium pyrophosphate, potassium tripolyphosphate, potassium pyrophosphate, or adenosine triphosphate. The powder can be reconstituted into a solution (e.g., by adding a buffer to the powder) prior to being applied to the substrate.

In another aspect, this document provides a method for removing a polyelectrolyte adhesive from a substrate where the polyelectrolyte adhesive comprises a polyamine, a polyphosphate, and a beneficial agent. The method can include, or consist essentially of, contacting the polyelectrolyte adhesive with a removal agent. The removal agent can be TRIS, phosphoric acid, 1M HCl, polyphosphate, EDTA, or NaCl. The removal agent can be a natural removal agent. The substrate can include staining, and removal of the polyelectrolyte adhesive also can include removal of the staining from the substrate.

In another aspect, this document provides a method for enhancing a polyelectrolyte adhesive coating on a substrate. The method can include, or consist essentially of applying a post treatment to a polyelectrolyte adhesive coating on a substrate. The post treatment can be a polyether (e.g., polyethylene glycol, poly propylene glycol, and poly butylene glycol). A polyether can have a molecular weight of about 50 daltons to about 10000 daltons. The post treatment can be a sweetener (e.g., sucralose and xylitol). The post treatment can be a flavoring agent (e.g., mint, thymol, menthol, and peppermint oil). The post treatment can be a rheology agent (e.g., laponite, gel white, glycerin, and poly glycols). The post treatment can enhance an appearance of the polyelectrolyte adhesive coating. The post treatment can increase the durability of the polyelectrolyte adhesive coating. The post treatment can protect the polyelectrolyte adhesive coating from degradation.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIGS. 1A-1G show polyamine and polyphosphate combinations in polyelectrolyte adhesives. FIG. 1A shows examples of polyamines: polyallylamine (PAA), a copolymer of primary amine and secondary amine (PAA-D), a co-polymer of secondary amine and sulfur dioxide with less chlorine content (PASA), and a co-polymer of secondary amine and sulfur dioxide (PAS). FIG. 1B shows examples of polyphosphates: polyphosphates sodium trimetaphosphate (STP), sodium hexametaphosphate (SHMP), tripolyphosphate (TPP), and pyrophosphate (PPi). FIGS. 1D-1G contain images of teeth before application of polyelectrolyte adhesives, teeth after application of polyelectrolyte adhesive coating, and teeth after brushing a coating of polyelectrolyte adhesive for 10 seconds with an electric toothbrush. FIG. 1C contains images of teeth with polyelectrolyte adhesive coatings containing polyamine PAA and polyphosphates STP, SHMP, TPP, and PPi. FIG. 1D contains images of teeth with polyelectrolyte adhesive coatings containing polyamine PAA polymers poly acrylic acid, poly aspartate, sodium phytate, and calcium pyrophosphate before application, after application and after brushing. FIG. 1E contains images of teeth with polyelectrolyte adhesive coatings containing polyamine PAA-D and polyphosphates STP, SHMP, TPP, and PPi. FIG. 1F contains images of teeth with polyelectrolyte adhesive coatings containing polyamine PAS and polyphosphates STP, SHMP, TPP, and PPi. FIG. 1G contains images of teeth with polyelectrolyte adhesive coatings containing polyamine PASA and polyphosphates STP, SHMP, TPP, and PPi.

FIGS. 2A-2B are images of teeth following application of polyelectrolyte adhesives and brushing with an electric toothbrush. FIG. 2A contains images of teeth with polyelectrolyte adhesive coatings containing ratios of PAA:STP at different pHs. FIG. 2B contains images of teeth with polyelectrolyte adhesive coatings containing ratios of PAA-D:STP at different pHs.

FIG. 3 contains images of teeth before application, after application of a polyelectrolyte adhesive containing adenosine tri phosphate (ATP), after exposure to saliva, and after brushing.

FIGS. 4A-4D show naturally-derived polyamines and anions for use in polyelectrolyte adhesives. FIG. 4A shows examples of naturally-derived polyamines: chitosan (deacetylated), spermine, spermidine, and putrescine. FIG. 4B shows examples of polyanions: poly acrylic acid, poly aspartate (sodium poly aspartate), sodium phytate, and calcium pyrophosphate. FIG. 4C shows polyelectrolyte adhesive preparations with polyamines including amines derived from natural sources prior to application. FIG. 4D contains images of teeth before application of a polyelectrolyte adhesive with polyamines including amines derived from natural sources, after application, after exposure to saliva, after exposure to staining, and after brushing.

FIGS. 5A-5B show polyelectrolyte adhesives with whitening agents. FIG. 5A shows polyelectrolyte adhesive preparations with whitening agents prior to application. FIG. 5B contains images of teeth prior to application of polyelectrolyte adhesive preparations with whitening agents and photographs of teeth coated with polyelectrolyte adhesive preparations with whitening agents before and after brushing.

FIGS. 6A-6B show polyelectrolyte adhesives with ZnO. FIG. 6A contains polyelectrolyte adhesive preparations with ZnO and reaction times. FIG. 6B contains images of teeth prior to application of polyelectrolyte adhesive coatings containing ZnO, after application, after exposure to saliva and scratching, after exposure to staining, and after brushing.

FIG. 7 contains images of teeth before and after application of a polyelectrolyte adhesive using applicator brushes.

FIG. 8 contains images of teeth before and after application of a polyelectrolyte adhesive using applicator strips.

FIGS. 9A-9B show lyophilized polyelectrolyte adhesives. FIG. 9A shows lyophilized polyelectrolyte adhesive formulations and preparations. FIG. 9B contains images of teeth prior to application of lyophilized polyelectrolyte adhesives, after application, after exposure to saliva, after exposure to staining, and after brushing.

FIG. 10 contains images of teeth before and after application of polyelectrolyte adhesive coatings, and after brushing and post treatment.

FIGS. 11A and 11B show polyelectrolyte adhesive coating removal conditions. FIG. 11A contains images of teeth coated with a polyelectrolyte adhesive before and after exposure to 2-(N-morpholino)ethanesulfonic acid (MES) paste, clinical pumice, tris(hydroxymethyl)aminomethane (TRIS) paste, sodium chloride (NaCl) paste, sodium carbonate (Na₂CO₃) paste, diluted (dil.) Na₂CO₃ paste, and dental scaling. FIG. 11B contains images of teeth coated with a polyelectrolyte adhesive before and after exposure to 0.1M HCl, 1M HCl, 3% phosphoric acid, 30% phosphoric acid, coarse pumice, and fine pumice.

FIG. 12 shows contains images of teeth before and after application of polyelectrolyte adhesive coatings containing a bioactive glass opacifier, an alumino silicate opacifier, or a mixed oxides opacifier, and after brushing in both wet and dry conditions.

FIG. 13 contains images of teeth before and after application of polyelectrolyte adhesive coatings having a PAA:TPP ratio of 1:1, after brushing, and after post treatment

### DETAILED DESCRIPTION

This document provides materials and methods for using a polyelectrolyte adhesive containing a conjugate of at least a polyamine (e.g., a naturally-derived polyamine and/or a synthetic polyamine), a polyphosphate, and one or more beneficial agents (e.g., TiO₂, ZnO, apatite, and/or mint oil) to adhere to a substrate (e.g., teeth, skin, hair, nails, hair, lips, and eyelashes). A substrate can be in vivo or in vitro. For example, a polyelectrolyte adhesive described herein (e.g., containing a conjugate of at least a polyamine, a polyphosphate, and one or more beneficial agents) can be used to adhere to a tooth, a tooth component, or an inorganic dental material (e.g., a human tooth, a human tooth component, or an inorganic dental material within a human's mouth). In some cases, this document provides materials and methods for adhering or attaching a polyelectrolyte adhesive containing one or more beneficial agents (e.g., one or more whitening agents) to teeth as a coating to provide a desired benefit (e.g., whiter appearance, more natural appearance, glossier appearance, flavor, breath freshening, and/or medicament).

In some cases, a coating (e.g., a coating including an opacifier such as bioactive glass, sodium silicate, or aluminum silicate) can be a clear coating. For example, a coating can be clear under wet conditions. For example, a coating can be clear under dry conditions.

In some cases, a coating (e.g., a coating including an opacifier such as bioactive glass, sodium silicate, or aluminum silicate) can be a coating that is not clear (e.g., white). For example, a coating can be not clear under wet conditions. For example, a coating can be not clear under dry conditions.

In some cases, a coating (e.g., a coating including an opacifier such as bioactive glass, sodium silicate, or aluminum silicate) can change from clear to not clear (e.g., white) or vice versa. In some cases, a coating that is clear can be not clear (e.g., white) under wet conditions. For example, a coating (e.g., a coating including an opacifier such as bioactive glass, sodium silicate, or aluminum silicate) can be clear under dry conditions (e.g., when applied to dry teeth), and can be white under wet conditions (e.g., when exposed to saliva). In some cases, a coating that is not clear (e.g., white) can be clear under wet conditions. For example, a coating (e.g., a coating including an opacifier such as bioactive glass, sodium silicate, or aluminum silicate) can be white under dry conditions (e.g., when applied to dry teeth), and can be clear under wet conditions (e.g., when exposed to saliva).

In some cases, this document provides materials and methods for contacting teeth with a polyelectrolyte adhesive containing a conjugate of at least a naturally-derived polyamine, a polyphosphate, and one or more whitening agents to provide the teeth with a coating that provides a whiter appearance. For example, this document provides materials and methods for contacting teeth with a polyelectrolyte adhesive containing a conjugate of at least a naturally-derived polyamine, a polyphosphate, and an opacifier (e.g., apatite) that provides a more natural appearance. For example, this document provides materials and methods for contacting teeth with a polyelectrolyte adhesive containing a conjugate of at least a naturally-derived polyamine, a polyphosphate, and a flavor (e.g., mint oil) that provides fresher breath. Polyelectrolyte adhesive coatings and dental benefits provided by polyelectrolyte adhesives provided herein can be maintained after brushing.

A polyelectrolyte adhesive containing a conjugate of at least a polyamine (e.g., a naturally-derived polyamine and/or a synthetic polyamine), a polyphosphate, and one or more beneficial agents (e.g., TiO₂, ZnO, apatite, and/or mint oil) can have the ability to interact with or bind to a tooth, a tooth component (e.g., enamel, hydroxyapatite, acquired dental pellicle, cementum, crown, cervix, cementoenamel junction, or apex), or inorganic dental materials (e.g., crowns, caps, braces, or fillings) using weak or strong ionic or covalent interactions to the surface of the tooth, tooth component, or inorganic dental material, thus attaching or adhering one or more beneficial agents to the tooth, tooth component, or inorganic dental material. A polyelectrolyte adhesive can adhere to organic or inorganic materials on the surface of a tooth, tooth component, or inorganic dental material. A tooth, tooth component, or inorganic dental material can be from a mammal (e.g., a human, dog, cat, cow, or horse).

A polyelectrolyte adhesive can be formed by contacting a polycation, a polyanion, and one or more beneficial agents (e.g., TiO₂, ZnO, apatite, and/or mint oil). For example, a polyelectrolyte adhesive can be formed by contacting a polyamine (e.g., a naturally-derived polyamine and/or a synthetic polyamine), a polyphosphate, and one or more beneficial agents (e.g., TiO2, ZnO, apatite, and/or mint oil). When forming a polyelectrolyte adhesive described herein, the polyamine, the polyphosphate, and the one or more beneficial agents can be provided separately, together, or in any combination. In cases where a polyamine, a polyphosphate, and one or more beneficial agents are provided separately, they can be provided simultaneously or serially. A polyamine, a polyphosphate, and one or more beneficial agents provided serially can be provided in any order. In some cases, a polyelectrolyte adhesive provided herein can be formed by contacting a first composition containing a polyphosphate (e.g., STP) to a second composition containing a polyamine (e.g., a naturally-derived polyamine such as chitosan) and a whitening agent (e.g., TiO₂). In some cases, a polyelectrolyte adhesive provided herein can be formed by contacting a first composition containing a polyphosphate (e.g., STP) and one or more beneficial agents to a second composition containing a polyamine (e.g., a naturally-derived polyamine such as chitosan). When forming a polyelectrolyte adhesive described herein, the polyamine, the polyphosphate, and the one or more beneficial agents can be provided in any form (e.g., a solution (e.g., an aqueous solution), powder (e.g., a lyophilized powder, powder formed by atomization, and powder formed by evaporation), paste (e.g., a soluble paste), gel (e.g., a hydrophobic gel, and gels formed by sol gel methods), or suspension (e.g., suspensions formed by mechanical mixing)). In cases where a polyelectrolyte adhesive described herein is provided as a powder, the powder can be resuspended and/or reconstituted into a solution prior to application. For example, when a polyelectrolyte adhesive described herein is provided as a powder, the powder can be resuspended and/or reconstituted into a solution by adding a buffer to the powder.

Any appropriate polyamine can be used to form a polyelectrolyte adhesive described herein. A polyamine can be a polymer containing repeating units of primary amines, secondary amines, amines mixed with other repeat units (e.g., such as sulfur dioxide), or combinations thereof. A polyamine can be a synthetic polyamine. Examples of synthetic polyamines that can be used to form a polyelectrolyte adhesive described herein include, without limitation, PAA, copolymers of a primary and secondary amine (e.g. PAA-D), copolymers of secondary amine and sulfur dioxide (e.g., PAS), PAA hydrochloride, DAA, polyampholite, polylysine, polyvinyl amine, PEI, heparin, and polymethacrylate (e.g., EUDRAGIT® E 100, EUDRAGIT® EPO, EUDRAGIT® RL30D, EUDRAGIT® RS30D, EUDRAGIT® L30D-55), polyhistidine. A polyamine can be a naturally-derived polyamine. As used herein a “naturally-derived polyamine” is any polyamine including amines derived from natural sources (e.g., animals such as crustaceans or plants such as soy, wheat, and rice). Examples of amines derived from natural sources that can be used to form a polyamine for use in a polyelectrolyte adhesive described herein include, without limitation, chitosan (e.g., deacetylated chitosan), soy-based polyamines, spermine, spermidine, putrescine, polyamine extracted from wheat germ (Triticum aestivum), and polyamine extracted from rice. A polyamine polymer described herein can be a hydrochloride salt form or a free base form. Additional examples of polyamines that can be used to form a polyelectrolyte adhesive described herein include, without limitation, those commercially available from Nittobo Medical Co., LTD (see, e.g., the PAS series, the PAA series, and the polyampholite series), Millipore Sigma, VWR, and polysciences. A polyamine described herein can be a homopolymer, copolymer, or a combination thereof. For example, a polyamine used to form a polyelectrolyte adhesive described herein can include in combination of one or more synthetic polyamines and one or more naturally-derived polyamines. In some cases, a polyamine used as described herein can have a molecular weight of less than about 200,000 daltons (e.g., less than about 100,000 daltons; less than about 50,000 daltons; less than about 30,000 daltons; less than about 15,000 daltons; less than about 10,000 daltons; less than about 1,000 daltons; or less than about 100 daltons). In some cases, a polyamine used as described herein can have a molecular weight of about 5,000 daltons to about 20,000 daltons. In some cases, a polyamine used as described herein can have a pH of about 4 to about 10 (e.g., about 6.5 to about 9, about 7 to about 9.5, or about 8 to about 9). A polyamine can be pH adjusted to have a pH of about 6 to about 10. For example, PAA can be pH adjusted to have a pH of about 9. For example, deacetylated chitosan can be pH adjusted to have a pH of about 4. For example, pH adjustment can be made using an acid (e.g., hydrochloric acid, acetic acid, and/or phosphoric acid).

A polyelectrolyte adhesive containing a conjugate of at least a polyamine, a polyphosphate, and one or more beneficial agents described herein can include from about 0.01 weight percent to about 10 weight percent of the polyamine (e.g., PAA and/or chitosan). In some cases, a dilute polyelectrolyte adhesive can be used, for example, as a mouth rinse, and can include about 0.01 wt % to about 1 wt % (e.g., from about 0.01 wt % to about 0.1 wt %from about 0.2 wt % to about 0.9 wt %, from about 0.3 wt % to about 0.8 wt %, from about 0.4 wt % to about 0.7 wt %, or from about 0.5 wt % to about 0.6 wt %) polyamine. In some cases, a concentrated polyelectrolyte adhesive can be used, for example, as a toothpaste or gel, and can include about 1 wt % to about 10 wt % (e.g., from about 2 wt % to about 9 wt %, from about 3 wt % to about 8 wt %, from about 4 wt % to about 7 wt %, from about or from about 5 wt % to about 6 wt %) polyamine. For example, a polyelectrolyte adhesive can include about 0.75 wt % PAA. For example, a polyelectrolyte adhesive can include about 0.01 wt % to about 5 wt % chitosan.

Any appropriate polyphosphate can be used to form a polyelectrolyte adhesive described herein. Any appropriate polyphosphate can be used to form a polyelectrolyte adhesive described herein. A polyphosphate can be a cyclic polyphosphate, a linear polyphosphate, a potassium salt of a polyphosphate, or a sodium phosphate salt of a polyphosphate. A polyphosphate can be a polymer having any phosphate-containing compound or salt thereof. Examples of polyphosphates that can be used as described herein include, without limitation, hexametaphosphate (e.g., SHMP), trimetaphosphate (e.g., STP), tripolyphosphate (e.g., TPP), pyrophosphate (e.g., PPi), potassium tripolyphosphate, potassium pyrophosphate, and adenosine triphosphate. Optionally, other poly-acids can also be used in place of or in addition to a polyphosphate in a polyelectrolyte adhesive described herein. Examples of poly-acids that can be used in a polyelectrolyte adhesive described herein include, without limitation, introduced such as alginates (e.g., alginic acid) crosslinked with CaCl₂, carrageenan, sodium alginate, polyacrylic acid, poly aspartate, sodium phytate, calcium pyrophosphate, polymers of oleic acid and glycerol such as those commercially available from ABITEC (see, e.g., the Caprol® series, and the Capmul® series), polymers of acrylic acid with allyl sucrose or allyl pentaerythritol such as those commercially available from Lubrizol (see, e.g., the Carbopol® polymers), polylactic-co-glycolic acid (PLGA), polystyrenesulfate, polyvinyl sulfate, polyglutamic acid, and/or polyaspartic acid. A polyphosphate used to form a polyelectrolyte adhesive described herein can be a homopolymer, copolymer, or a combination thereof. In some cases, a polyphosphate used as described herein can have a molecular weight greater less than about 50,000 daltons (e.g., less than about 20,000 daltons; less than about 5,000 daltons; less than about 3,000 daltons; less than about 2,000 daltons; or less than about 1,000 daltons). In some cases, a polyphosphate can aid in enamel remineralization. In some cases, a polyphosphate or poly-acid can aid in thickening and/or emulsification of a polyelectrolyte adhesive.

A polyelectrolyte adhesive containing a conjugate of at least a polyamine (a naturally-derived polyamine and/or a synthetic polyamine), a polyphosphate, and one or more beneficial agents described herein can include from about 0.05 wt % to about 25 wt % of polyphosphate (e.g., STP, SHMP, TPP, or PP). In some cases, a dilute polyelectrolyte adhesive can be used, for example, as a mouth rinse, and can include about 0.05 wt % to about 1 wt % (e.g., from about 0.1 wt % to about 0.9 wt %, from about 0.15 wt % to about 0.8 wt %, from about 0.2 wt % to about 0.7 wt %, or from about 0.25 wt % to about 0.6 wt %) polyphosphate. In some cases, a concentrated polyelectrolyte adhesive can be used, for example, as a toothpaste or gel, and can include about 1 wt % to about 25 wt % (e.g., from about 2 wt % to about 20 wt %, from about 3 wt % to about 15 wt %, from about 4 wt % to about 10 wt %, or from about 5 wt % to about 8 wt %) polyphosphate. In some cases, the wt % of the polyphosphate can be comparable to the wt % of the polyamine. In some cases, the wt % of the polyphosphate can be higher (e.g., 2 times (2×) higher, 3× higher, 5× higher, 7× higher, 10× higher, or 20× higher) than the wt % of the polyamine.

In some cases, a polyelectrolyte adhesive containing a conjugate of at least a polyamine, a polyphosphate, and one or more beneficial agents described herein can include a w/w ratio of polyamine (e.g., PAA and/or chitosan) to polyphosphate (e.g., STP, SHMP, TPP, and/or PP) of about 5:1 to about 1:20 (e.g., about 2:1 to about 1:18, about 1:1 to about 1:15, about 1:1 to about 1:12, about 1:1 to about 1:10, about 1:2 to about 1:9, about 1:3 to about 1:8, or about 1:5 to about 1:8). For example, a polyelectrolyte adhesive described herein can include a w/w ratio of polyamine to polyphosphate of about 10:1. For example, a polyelectrolyte adhesive described herein can include a w/w ratio of polyamine to polyphosphate of about 5:1. For example, a polyelectrolyte adhesive described herein can include a w/w ratio of polyamine to polyphosphate of about 3:1. For example, a polyelectrolyte adhesive described herein can include a w/w ratio of polyamine to polyphosphate of about 1:1. For example, a polyelectrolyte adhesive described herein can include a w/w ratio of polyamine to polyphosphate of about 1:3. For example, a polyelectrolyte adhesive described herein can include a w/w ratio of polyamine to polyphosphate of about 1:5. For example, a polyelectrolyte adhesive described herein can include a w/w ratio of polyamine to polyphosphate of about 1:6.7. For example, a polyelectrolyte adhesive described herein can include a w/w ratio of polyamine to polyphosphate of about 1:10.

Any appropriate method can be used to make a polyelectrolyte adhesive containing a conjugate of at least a polyamine, a polyphosphate, and one or more beneficial agents described herein, and having the ability to interact with and/or bind to tooth, tooth component, or inorganic dental material. In some cases, a polyamine and/or a polyphosphate can be obtained from a commercial source or can be synthesized from the polymerization of a desired monomer or combination of different monomers. In some cases, standard polymer synthesis techniques (e.g., addition polymerization, sol-gel synthesis, phase separation, template-assisted synthesis, step growth polymerization, or co-polymerization using chemical or photochemical radical synthesis) can be used to produce polyamines and/or polyphosphates.

Any appropriate beneficial agent can be used to form a polyelectrolyte adhesive described herein. As used herein, a beneficial agent can be any agent that provides a tooth, tooth component, or inorganic dental material with a desired benefit (e.g., whiter appearance, more natural, hardening, stain resistance, appearance, flavor, breath freshening, and/or medicament). Examples of beneficial agents that can be included in a polyelectrolyte adhesive described herein include, without limitation, whitening agents, opacifiers, rigidity promoters, flavoring agents, antimicrobials, and coolants.

In cases where a beneficial agent is a whitening agent, any appropriate whitening agent can be used to form a polyelectrolyte adhesive described herein. Examples of whitening agents that can be used in a polyelectrolyte adhesive described herein include, without limitation, particles composed of TiO₂, ZnO, hydroxyapatite (HA), silicon dioxide (SiO₂), bioactive glass, aluminum oxide, zirconium oxide, sodium silicate, zirconium silicate, alumino silicate, and/or calcium phosphate. See, e.g., Schilling et al., Photochem. Photobiol. Sci., 9:495-509 (2010); and U.S. Pat. No. 6,004,567. Particulate whitening agents can range in size from about 5 nanometer (nm) to about 1000 nm (1 pm) in size (e.g., from about 10 nm to about 900 nm, from about 25 nm to about 750 nm, from about 50 nm to about 500 nm, from about 75 nm to about 450 nm, from about 100 nm to about 400 nm, from about 125 nm to about 350 nm, or from about 150 nm to about 300 nm). In some cases, a polyelectrolyte adhesive containing a conjugate of at least a polyamine, a polyphosphate, and one or more whitening agents described herein can include two or more whitening agents (e.g., two whitening agents, three whitening agents, four whitening agents, or five whitening agents). For example, a polyelectrolyte adhesive can include TiO₂ and HA; TiO₂ and ZnO; TiO₂ and SiO₂; HA and SiO₂; or TiO₂, HA, SiO₂, and ZnO. A whitening agent can be covalently or non-covalently attached to any component of a polyelectrolyte adhesive such as a polyamine and/or a polyphosphate. A polyelectrolyte adhesive containing a conjugate of at least a polyamine, a polyphosphate, and one or more whitening agents described herein can include from about 0.005 percent to about 20 percent whitening agent. For example, a polyelectrolyte adhesive described herein can include from about 0.005 percent to about 10 percent (e.g., from about 0.005% to about 9%, from about 0.005% to about 8%, from about 0.005% to about 7%, from about 0.005% to about 6%, from about 0.005% to about 5%, from about 0.005% to about 4%, from about 0.005% to about 3%, from about 0.005% to about 2%, from about 0.005% to about 1%, from about 0.005% to about 0.5%, from about 0.005% to about 0.1%, from about 0.05% to about 10%, from about 0.5% to about 10%, from about 1% to about 10%, from about 2% to about 10%, from about 5% to about 10%, from about 8% to about 10%, from about 0.05% to about 8%, from about 0.5% to about 5%, from about 1% to about 3%, from about 3% to about 5%, from about 5% to about 8%, from about 0.07% to about 1.9%, from about 0.09% to about 1.8%, or from about 1% to about 1.5%) TiO₂. For example, a polyelectrolyte adhesive described herein can include from about 0.5 percent to about 50 percent (e.g., from about 0.5% to about 40%, from about 0.5% to about 30%, from about 0.5% to about 20%, from about 0.5% to about 10%, from about 0.5% to about 8%, from about 0.5% to about 5%, from about 0.5% to about 3%, from about 0.5% to about 1%, from about 2% to about 50%, from about 5% to about 50%, from about 7% to about 50%, from about 10% to about 50%, from about 20% to about 50%, from about 30% to about 50%, from about 40% to about 50%, from about 1% to about 40%, from about 5% to about 30%, from about 10% to about 20%, from about 20% to about 30%, from about 30% to about 40%, from about 0.7% to about 9%, from about 0.9% to about 8%, or from about 1% to about 7%) ZnO. For example, a polyelectrolyte adhesive described herein can include from about 1 percent to about 30 percent (e.g., from about 2% to about 19%, from about 3% to about 18%, from about 4% to about 17%, from about 8% to 30%, or from about 5% to about 15%) HA.

In cases where a beneficial agent is an opacifier, any appropriate opacifier can be used to form a polyelectrolyte adhesive described herein. As used herein, an “opacifier” can be any agent that alters the interaction between light and a polyelectrolyte adhesive coating described herein. In some cases, an opacifier can block or distort (e.g., refract) the passage of light through the coating. In some cases, an opacifier can distort the reflection of light by the coating. An opacifier can be a light diffusing agent (e.g., a mineral). Examples of opacifiers that can be used in a polyelectrolyte adhesive described herein include, without limitation, apatite, hydroxyapatite, mica, titanated mica, zinc oxide, zirconium oxide, aluminum oxide, boron trioxide, bioactive glass, sodium oxide, and silicates (e.g., sodium silicates, aluminum silicates, or zirconium silicates). In some cases, a polyelectrolyte adhesive described herein including an opacifier can be used to provide teeth with a coating that provides a more natural appearance. A more natural appearance can be evaluated by the surface of a tooth having a heterogeneous (e.g., having which allows for shading and/or spacing amongst diverse shades or hues of white and off-white colors) white color, luster, sheen, gloss, and/or translucency. A natural appearance (e.g., translucency) can be measured using, for example, a spectrophotometer as described elsewhere (see, e.g., “Relative translucency of ceramic systems for porcelain veneers,” by Karine Tenorio Landim Barizon). An opacifier can have any appropriate particle size. In some cases, an opacifier can have a particle size from about 40 nm to about 500 nm in size (e.g., from about 45 nm to about 450 nm, from about 50 nm to about 400 nm, from about 55 nm to about 375 nm, from about 60 nm to about 350 nm, from about 65 nm to about 325 nm, from about 70 nm to about 315 nm, or from about 75 nm to about 300 nm).

In cases where a beneficial agent enhances hardness, any appropriate rigidity promoter can be used to form a polyelectrolyte adhesive described herein. As used herein, a “rigidity promoter” can be any agent that enhances the hardness of a polyelectrolyte adhesive provided herein such that a polyelectrolyte adhesive coating is more resistant to mechanical abrasion (e.g., scratching from toothbrushes, toothpicks, and hard foods). Examples of rigidity promoters that can be used in a polyelectrolyte adhesive described herein include, without limitation, ZnO, chitosan, carrageenan, oxides (e.g., zirconium oxide and aluminum oxide), fluoroapatite, and silicates. A polyelectrolyte adhesive containing a conjugate of at least a polyamine, a polyphosphate, and one or more beneficial agents described herein can include from about 0.05 percent to about 50 percent rigidity promoter. For example, a polyelectrolyte adhesive described herein can include from about 0.5 percent to about 10 percent (e.g., from about 0.05% to about 40%, from about 0.05% to about 30%, from about 0.05% to about 20%, from about 0.05% to about 10%, from about 0.05% to about 8%, from about 0.05% to about 5%, from about 0.05% to about 3%, from about 0.05% to about 1%, from about 0.5% to about 50%, from about 1% to about 50%, from about 5% to about 50%, from about 7% to about 50%, from about 10% to about 50%, from about 20% to about 50%, from about 30% to about 50%, from about 40% to about 50%, from about 0.5% to about 40%, from about 1% to about 30%, from about 5% to about 20%, from about 7% to about 15%, from about 1% to about 10%, from about 10% to about 20%, from about 20% to about 30%, from about 0.7% to about 9%, from about 0.9% to about 8%, or from about 1% to about 7%) ZnO. In some cases, a polyelectrolyte adhesive described herein can include about 2% ZnO.

In cases where a beneficial agent enhances stain resistance, any appropriate stain resistance agent can be used to form a polyelectrolyte adhesive described herein. As used herein, a “stain resistance agent” can be any agent that provides a tooth surface with an anionic charge and/or hydrophobicity. Examples of stain resistance agents that can be used in a polyelectrolyte adhesive described herein include, without limitation, silane coated oxides, polymers of acrylic acid with allyl sucrose or allyl pentaerythritol such as those commercially available from Lubrizol (see, e.g., the Carbopol® polymers), magnesium stearate, silanes, silicates, hydrophobic lipids, and amphiphilic lipids.

In cases where a beneficial agent is a flavoring agent, any appropriate flavoring agent can be used to form a polyelectrolyte adhesive described herein. A flavoring agent can be a natural flavoring agent or an artificial flavoring agent. Examples of flavoring agents that can be used in a polyelectrolyte adhesive described herein include, without limitation, mint (e.g., spearmint, peppermint, and wintergreen oils), sweeteners (e.g., sorbitol, sucralose, sodium saccharin, and xylitol), fruit-flavors (e.g., citrus oils), spices (e.g., cinnamon oil), and vanilla.

A flavoring agent can provide an electrolyte adhesive described herein with immediate and/or long lasting flavor. For example, an electrolyte adhesive including a flavoring agent can provide an immediate flavor burst lasting no more than about 10 minutes (e.g., lasting no more than 6 minutes, lasting no more than 5 minutes, lasting no more than 4 minutes, lasting no more than 3 minutes, lasting no more than 2 minutes, or lasting no more than 1 minute) after application. For example, an electrolyte adhesive including a flavoring agent can provide flavor lasting at least 0.5 hours (e.g., lasting at least 1 hour, lasting at least 1.5 hours, or lasting at least 2 hours). In some cases, an electrolyte adhesive including peppermint oil can provide an immediate burst lasting about 5 minutes, and can provide flavor lasted about 2 hours. A flavoring agent can provide an electrolyte adhesive described herein with a flavor level of about 0.1% to about 5%. For example, a flavoring agent can provide an electrolyte adhesive described herein with a flavor level of about 0.1%.

In cases where a beneficial agent is an antimicrobial agent, any appropriate antimicrobial agent can be used to form a polyelectrolyte adhesive described herein. Examples of antimicrobial agents that can be used in a polyelectrolyte adhesive described herein include, without limitation, antiseptics (e.g., chlorhexidine gluconate, cetylpyridinium chloride, triclosan, betadine), and antibiotics (e.g., penicillins, cephalosporins, ZnO, triclosan, zinc chloride).

In cases where a beneficial agent is a coolant, any appropriate coolant can be used to form a polyelectrolyte adhesive described herein. A coolant can be a natural coolant or a synthetic coolant. Examples of coolants that can be used in a polyelectrolyte adhesive described herein include, without limitation, menthol, carboxamide cooling agents, menthol isomers, p-menthane alcohols, and camphor.

A polyelectrolyte adhesive containing a conjugate of at least a polyamine, a polyphosphate, and one or more beneficial agents described herein also can contain one or more other molecules. For example, a polyelectrolyte adhesive also can be combined with a therapeutic agent (e.g. fluoride and/or anti-sensitivity compounds), remineralization particles (e.g., HA, fluoride, calcium, magnesium, phosphate, iron, and/or tin ions, and any salt forms thereof), astringent salts, odor neutralizers, anti-gingivitis agents, anti-plaque agents, anti-tartar agents, or any combination thereof.

This document also provides methods for applying a polyelectrolyte adhesive containing a conjugate of at least a polyamine, a polyphosphate, and one or more beneficial agents described herein. In some cases a polyelectrolyte adhesive can be formed and then applied directly to a tooth, tooth component, or inorganic dental material in a single step.

A polyelectrolyte adhesive containing a conjugate of at least a polyamine, a polyphosphate, and one or more beneficial agents described herein can be applied under conditions that allow a beneficial agent to adhere directly or indirectly to a tooth, tooth component, or inorganic dental material to provide the desired benefit (e.g., whiter appearance, more natural appearance, flavor, breath freshening, and/or medicament) to the tooth, tooth component, or inorganic dental material. For example, a polyelectrolyte adhesive containing a conjugate of at least a polyamine, a polyphosphate, and one or more beneficial agents provided herein can be applied to a tooth, tooth component, or inorganic dental material under dry or wet conditions. In some cases, a polyelectrolyte adhesive can be applied under typical conditions found in the mouth (e.g., the presence of saliva). For example, a polyelectrolyte adhesive can be applied to a tooth, tooth component, or inorganic dental material in the presence of saliva to alter the appearance of the tooth, tooth component, or inorganic dental material.

Any appropriate formulation can be used to deliver a polyelectrolyte adhesive containing a conjugate of at least a polyamine, a polyphosphate, and one or more beneficial agents provided herein to a tooth, tooth component, or inorganic dental material. For example, a polyelectrolyte adhesive provided herein can be incorporated into a paste (e.g., toothpaste), a mouth wash, a mouth rinse, an ingestable substance such as a drink or a food product, and oral and/or topical uses such as gum (e.g., chewing gum), gels (an application gel), powders, or creams. For example, a polyelectrolyte adhesive provided herein can be delivered using a brush (e.g., a brush with synthetic fibers such as polypropylene or nylon) and brushed onto a tooth, tooth component, or inorganic dental material. For example, a polyelectrolyte adhesive provided herein can be delivered on an applicator strip (e.g., a wax or plastic strip, such as a polyethylene strip) and applied directly to a tooth, tooth component, or inorganic dental material. Applicator strips containing a polyelectrolyte adhesive provided herein can be dissolvable strips. Applicator strips containing a polyelectrolyte adhesive provided herein can include a detachable backing. For example, a polyelectrolyte adhesive provided herein can be delivered in a dental tray or a mouth dam fit to a mammal's (e.g., a human's) tooth structure. In some cases, a formulation including a polyelectrolyte adhesive provided herein can include one or more pharmaceutical excipients. For example, a toothpaste containing a polyelectrolyte adhesive described herein can include one or more thickeners (e.g., polymers of oleic acid and glycerol such as those commercially available from ABITEC (see, e.g., the Caprol® series, and the Capmul® series), emulsifiers, cellulose derivatives, polyvinylpyrollidones, mineral colloids, or polyethylene glycol (PEG)), buffers, surfactants, fluorides, flavorings (e.g., peppermint, spearmint, wintergreen, or bubble gum), sweeteners (e.g., sucralose or xylitol), sugar alcohols (e.g., sorbitol, glycerol, or xylitol), sensitivity reducers (e.g., potassium nitrate), humectants (e.g., glycerin), and/or anti-bacterial agents (e.g., ZnO, Triclosan, and/or zinc chloride) that do not interfere with providing a beneficial feature (e.g., whitening) to a tooth, tooth component, or inorganic dental material.

An effective amount of a polyelectrolyte adhesive containing a conjugate of at least a polyamine, a polyphosphate, and one or more beneficial agents provided herein can be any appropriate amount that provides a beneficial feature to a tooth, tooth component, or inorganic dental material without inducing significant toxicity. It will be appreciated that the amount can be higher for certain formulations, e.g., mouthwash. In some cases, a polyelectrolyte adhesive containing a conjugate of at least a polyamine, a polyphosphate, and one or more whitening agents provided herein can be applied to a tooth, tooth component, or inorganic dental material to give the tooth, tooth component, or inorganic dental material a whiter appearance. In some cases, a polyelectrolyte adhesive containing a conjugate of at least a polyamine, a polyphosphate, and one or more opacifiers provided herein can be applied to a tooth, tooth component, or inorganic dental material to give the tooth, tooth component, or inorganic dental material a more natural appearance.

In some cases, a polyelectrolyte adhesive containing a conjugate of at least a polyamine, a polyphosphate, and one or more beneficial agents provided herein can be applied to a tooth, tooth component, or inorganic dental material to reduce and/or prevent staining. For example, a polyelectrolyte adhesive provided herein can serve as a protective shield against extrinsic and intrinsic stains, preventing new stains from forming on a tooth. For example, a polyelectrolyte adhesive provided herein can reduce the intensity of any stain previously found on a tooth.

In some cases, a polyelectrolyte adhesive containing a conjugate of at least a polyamine, a polyphosphate, and one or more beneficial agents provided herein can be applied to a tooth, tooth component, or inorganic dental material to protect and/or to repair enamel.

In some cases, a polyelectrolyte adhesive containing a conjugate of at least a polyamine, a polyphosphate, and one or more beneficial agents provided herein can be applied to a tooth, tooth component, or inorganic dental material to protect a substrate. For example, a polyelectrolyte adhesive provided herein can provide a substrate with a clear coating or a glossy coating that protects the substrate. For example, a polyelectrolyte adhesive containing a conjugate of at least a polyamine, a polyphosphate, and one or more beneficial agents such as sodium silicate, alumino silicate, bioactive glass, and/or a flavoring agent (e.g., mint, thymol, menthol, and peppermint oil) can provide a substrate with a clear coating or a glossy coating that protects the substrate

In some cases, a polyelectrolyte adhesive containing a conjugate of at least a polyamine, a polyphosphate, and one or more beneficial agents provided herein can be applied to a tooth, tooth component, or inorganic dental material for a period of time prior to washing, rinsing, or removal such that a desired benefit (e.g., whiter appearance, more natural appearance, flavor, breath freshening, and/or medicament) is provided to the tooth, tooth component, or inorganic dental material. For example, a toothpaste or other formulation configured to include a polyelectrolyte adhesive containing a conjugate of at least a polyamine, a polyphosphate, and one or more beneficial agents as described herein can be applied (e.g., applied directly or formed on) to a tooth, tooth component, or inorganic dental material and remain in contact with that tooth, tooth component, or inorganic dental material, without rinsing, for from about 30 seconds to about 60 minutes (e.g., from about 30 seconds to about 45 minutes, from about 30 seconds to about 30 minutes, from about 30 seconds to about 25 minutes, from about 1 minute to about 20 minutes, from about 2 minutes to about 15 minutes, from about 3 minutes to about 10 minutes, or from about 4 minutes to about 6 minutes). In some cases such as with a mouth wash, mouth rinse, or application gel, a polyelectrolyte adhesive can be allowed to be in contact with a tooth, tooth component, or inorganic dental material for a period of time for the composition to saturate the tooth, tooth component, or inorganic dental material.

In some cases, a tooth, tooth component, or inorganic dental material (e.g., a human's tooth) can be prepared prior to applying a polyelectrolyte adhesive containing a conjugate of at least a polyamine, a polyphosphate, and one or more beneficial agents provided herein. For example, a tooth, tooth component, or inorganic dental material can be washed, brushed, or polished (e.g., polished with pumice) prior to applying a polyelectrolyte adhesive provided herein. In some cases, the surface of the tooth, tooth component, or inorganic dental material can be treated with one or more agents capable of exposing calcium phosphate binding sites. For example, a tooth, tooth component, or inorganic dental material to be treated with a polyelectrolyte adhesive provided herein can be contacted with EDTA or phosphoric acid to expose calcium phosphate binding sites present on the tooth, tooth component, or inorganic dental material. In the case of phosphoric acid treatment, only tooth enamel can be exposed to the acid to prevent or reduce the risk of soft tissue damage.

In some cases, a polyelectrolyte adhesive containing a conjugate of at least a polyamine, a polyphosphate, and one or more beneficial agents provided herein can be enhanced after application to a tooth, tooth component, or inorganic dental material (e.g., a human's tooth). For example, a polyelectrolyte adhesive provided herein, after application to a tooth, tooth component, or inorganic dental material, can be enhanced by contacting a polyelectrolyte adhesive (e.g., a polyelectrolyte adhesive coating on a substrate) with a post treatment. In some cases, a post treatment can be a naturally-derived post treatment. A “naturally-derived post treatment” is any post treatment derived from natural sources (e.g., plants such as mint; and earth such as a clay like laponite). Examples of post treatments can include, without limitation, TRIS (e.g., 18.1M TRIS and 0.181M TRIS), H₃PO₄ (e.g., 3% H₃PO₄ and 0.3% H₃PO₄), acrylic polymer (e.g., 5% acrylic polymer), STP (e.g., 25% STP), calcium pyrophosphate (e.g., 5% calcium pyrophosphate), phytate (e.g., 25% phytate), aspartate polymer (e.g., 5% aspartate polymer), glycols, polyethers (e.g., polyethylene glycol, poly propylene glycol, and poly butylene glycol), sweeteners (e.g., sucralose and xylitol), rheology agents (e.g., laponite, gel white, glycerin, and poly glycols) and flavoring agents (e.g., mint). In some cases, a post-treatment can enhance an appearance (e.g., by providing a whiter appearance, a more natural appearance, and/or a glossy appearance) of a polyelectrolyte adhesive coating. In some cases, a post-treatment can increase the durability of a polyelectrolyte adhesive coating. In some cases, a post-treatment can protect the polyelectrolyte adhesive from degradation.

In some cases, an assay can be performed to confirm that a polyelectrolyte adhesive containing a conjugate of at least a polyamine, a polyphosphate, and one or more beneficial agents provided herein or a component of a composition provided herein has binding affinity for a tooth, tooth component, or inorganic dental material. For example, a material to be tested can be incubated with a HA matrix, and the amount of material in solution after HA binding can be compared with the initial concentration to determine, by difference, the amount of bound material. See, e.g., Raj et al., J. Biol. Chem., 267:5968-5976 (1992). In some cases, the HA bound material can be directly measured after dissolving the HA matrix with EDTA (Lamkin et al., J. Dent. Res., 75:803-808 (1996)). In some cases, an assay can be performed with an HA matrix that was pre-incubated with human saliva to coat the HA with proteins as described elsewhere (Lamkin et al., J. Dent. Res., 75:803-808 (1996)). In such cases, unbound saliva proteins can be removed by washing since their presence may interfere with the polyelectrolyte concentration determinations.

Any appropriate method can be used to assess the affinity of a polyelectrolyte adhesive containing a conjugate of at least a polyamine, a polyphosphate, and one or more beneficial agents provided herein for a tooth, tooth component, inorganic dental material, or an HA matrix. In some cases, a polyelectrolyte adhesive provided herein can be assessed for the ability to bind in vitro to a human tooth or a human tooth component. The tooth or tooth component can be subjected to different degrees of cleaning, such as brushing or polishing with pumice. The tooth or tooth component can then be treated with human saliva to form the acquired dental pellicle and incubated with a polyelectrolyte adhesive provided herein in the presence and absence of saliva. The binding to the tooth or tooth component can be determined by measuring the degree of whiteness.

Any appropriate method can be used to assess a polyelectrolyte adhesive containing a conjugate of at least a polyamine, a polyphosphate, and one or more beneficial agents provided herein for the ability to provide a desired benefit to a tooth, tooth component, or inorganic dental material. For example, visual inspection techniques 1 0 can be used to determine whether or not a polyelectrolyte adhesive provided herein can alter the appearance of a tooth, tooth component, or inorganic dental material. Such visual inspection techniques can include using shade guides for comparison as described elsewhere (Paravina et al., J. Esthet. Restor. Dent., 19:276-283 (2007)). In some cases, the ability of a polyelectrolyte adhesive provided herein to alter the appearance of a tooth, tooth component, or inorganic dental material (e.g., to make a tooth, tooth component, or inorganic dental material appear whiter) can be measured using reflectance spectrophotometry. In such cases, the tooth, tooth component, or inorganic dental material can be illuminated with a white light source and analyzed as to the amount of light absorbed at different wavelengths by reflectance spectrophotometry (colorimetry). These measurements can then be repeated with the UV light filtered from the light source. The difference in the reflectance values obtained with the inclusion and exclusion of UV light is the UV fluorescence spectrum of the tooth surface (see, e.g., Park et al., Dental Materials, 23:731-735 (2007)).

A polyelectrolyte adhesive containing a conjugate of at least a polyamine, a polyphosphate, and one or more beneficial agents provided herein can be removed from a tooth, tooth component, or inorganic dental material. For example, a polyelectrolyte adhesive can be removed from a substrate by contacting the adhered polyelectrolyte adhesive with a removal agent. A removal agent can be natural or synthetic. In some cases, a removal agent can have a high ionic strength. Examples of agents that can be used to remove a polyelectrolyte adhesive coating from a tooth, tooth component, or inorganic dental material include, without limitation, TRIS, phosphoric acid, polyphosphates, and NaCl. In some cases, a removal agent can be included in a composition (e.g., a removal composition). For example, a removal composition can include one or more removal agents (e.g., TRIS, phosphoric acid, and/or NaCl) and one or more additional components. Examples of additional components that can be included in a removal composition include, without limitation, buffers (e.g., MES buffers), acids (e.g., hydrochloric acid), polyphosphate, and chelating agents (e.g., EDTA). For example, a removal composition can include TRIS, MES, phosphoric acid, 1M HC1, polyphosphate, NaCl, and EDTA. In some cases, a substrate can have stains (e.g., extrinsic stains) prior to application of a polyelectrolyte adhesive described herein, and removal of the polyelectrolyte adhesive can be effective to remove stains from the substrate. In some cases, a substrate can have stains (e.g., extrinsic stains) prior to application of a polyelectrolyte adhesive described herein, and removal of the polyelectrolyte adhesive can be effective to improve the appearance of the substrate.

The invention will be further described in the following examples, which do not limit the scope of the invention described in the claims.

### EXAMPLES

### Example 1: Preparation of Polyelectrolyte Adhesive

### Polyamine and Polyphosphate Combinations

Polyelectrolyte adhesives containing various polyamines and various polyphosphates were prepared and applied to teeth.

Two teeth were placed in 10 mL of phosphate buffered solution (PBS) or water for 5-10 minutes. 1 mL of 1% TiO₂ (aq.) was added and mixed for 5-10 minutes. 10 mL of 5% polyamine at the required pH, and 10 ml of 5 wt % phosphate solution were added and mixed for 30 minutes. The teeth were taken out of the solution and rinsed with water before brushing for 10 seconds in water using an Oral-B® electric spin toothbrush to test the resistance of the coating.

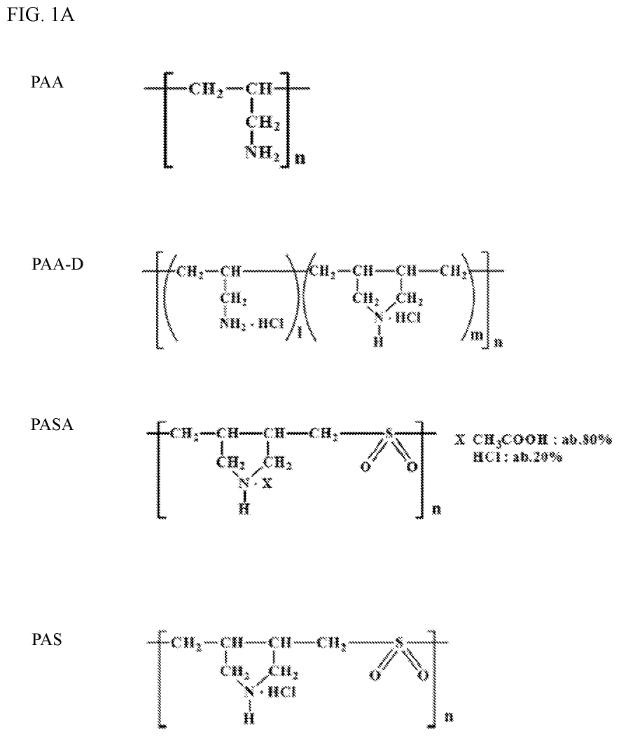

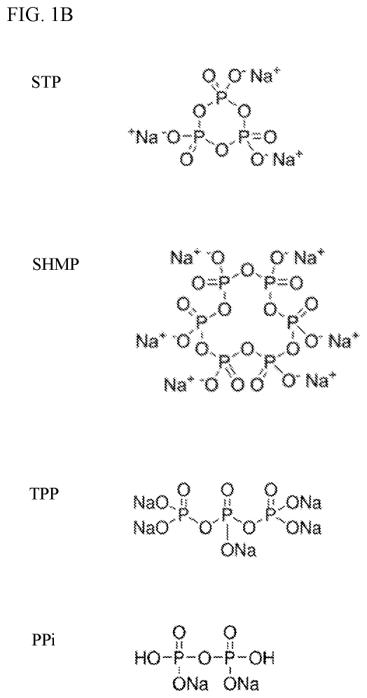

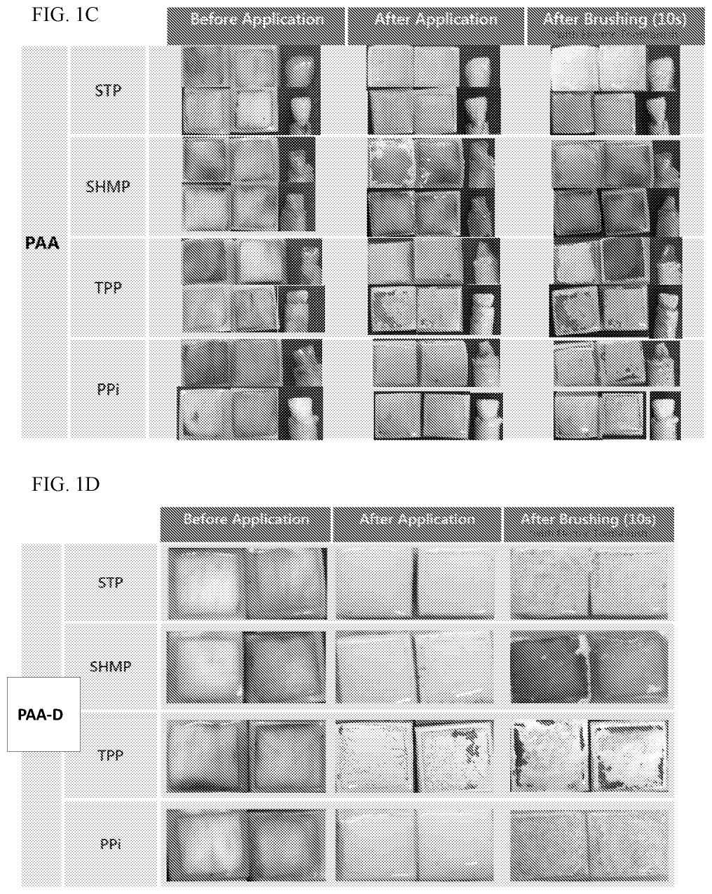

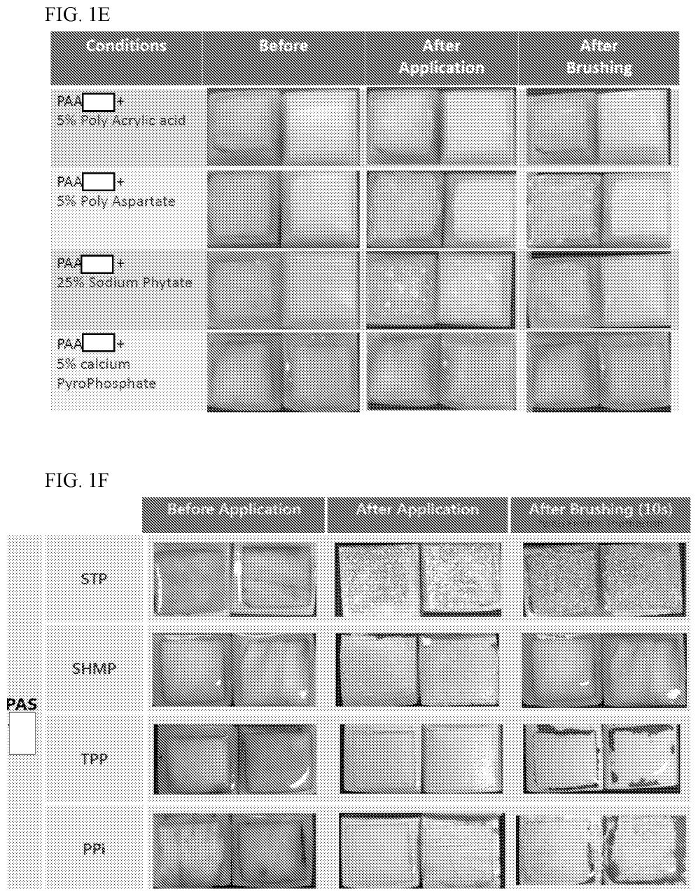

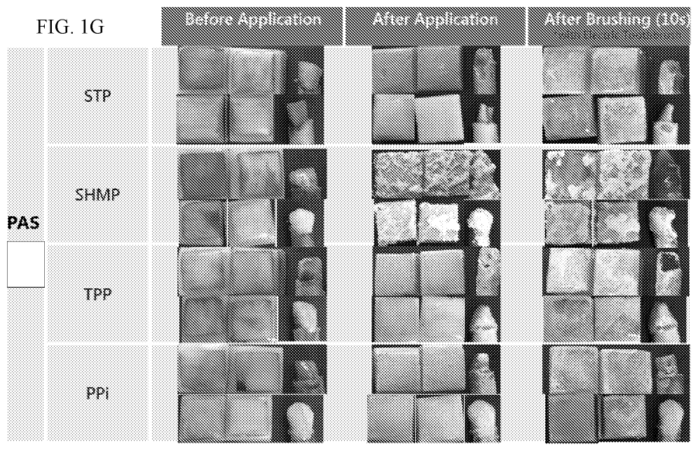

Polyelectrolyte adhesive coatings containing the polyamine PAA and various polyphosphates are shown before application, after application and after brushing in FIG. 1C. Polyelectrolyte adhesive coatings containing the polyamine PAA and various polymers are shown before application, after application and after brushing in FIG. 1D. Polyelectrolyte adhesive coatings containing the polyamine PAA-D and various polyphosphates are shown before application, after application and after brushing in FIG. 1E. Polyelectrolyte adhesive coatings containing the polyamine PAS and various polyphosphates are shown before application, after application and after brushing in FIG. 1F. Polyelectrolyte adhesive coatings containing the polyamine PASA and various polyphosphates are shown before application, after application and after brushing in FIG. 1G.

### Polyamine: Polyphosphate Ratios and pH

Polyelectrolyte adhesives containing various ratios of polyamine:polyphosphate were prepared and applied to teeth at varying pH.

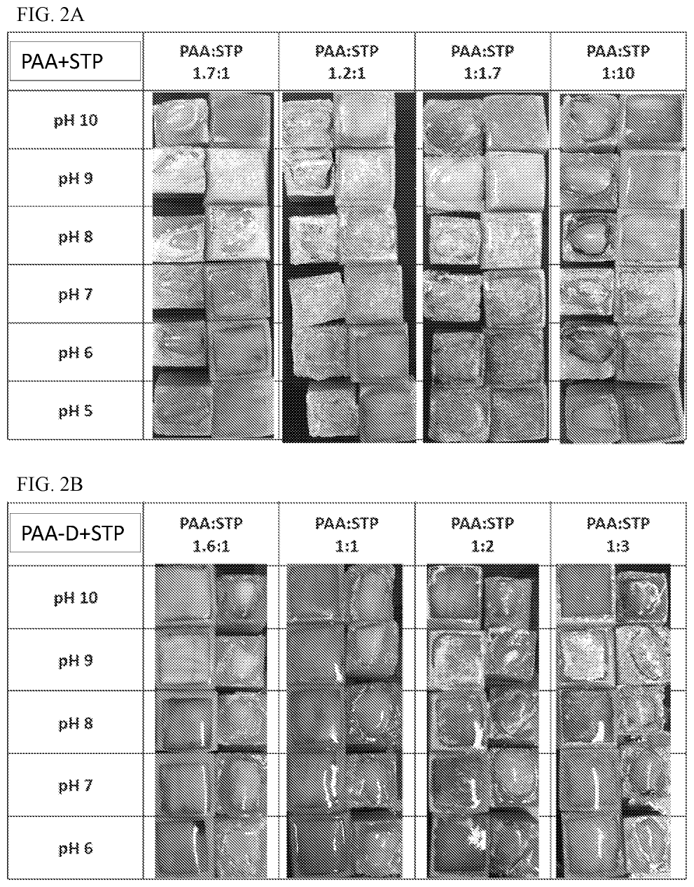

Polyelectrolyte adhesive coatings containing ratios of PAA:STP at different pHs are shown before application, after application and after brushing in FIG. 2A. Polyelectrolyte adhesive coatings containing the polyamine PAA-D:STP at different pHs are shown before application, after application and after brushing in FIG. 2B.

### Adenosine Tri Phosphate (ATP)

Polyelectrolyte adhesives containing ATP were prepared and applied to teeth. An exemplary formulation is shown in the table below.

| Materials (in mL) | |
|---|---|
| H2O | 4.05 mL |
| PAA (pH~9) | 0.75 mL |
| 1% TiO2 | 0.2 mL |
| 10% ATP | 5 mL |

Two teeth were placed in 10 mL of phosphate buffered solution (PBS) or water for 5-10 minutes. 1 mL of 1% TiO₂ (aq.) was added and mixed for 5-10 minutes. 10 mL of 5% polyamine at the required pH, and 10 ml of 5 wt % phosphate solution were added and mixed for 30 minutes. The teeth were taken out of the solution, incubated in artificial saliva for 1 hour, and brushed with a tooth brush for 30 seconds.

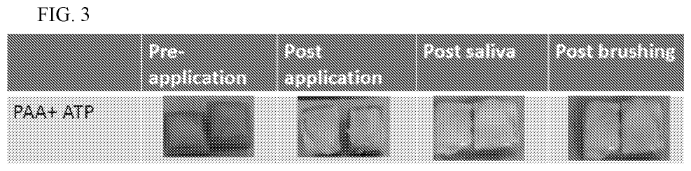

Polyelectrolyte adhesive coatings containing ATP are shown before application, after application, after exposure to saliva, and after brushing in FIG. 3.

Natural amine sources

Polyelectrolyte adhesives containing polyamines including amines derived from natural sources were prepared and applied to teeth.

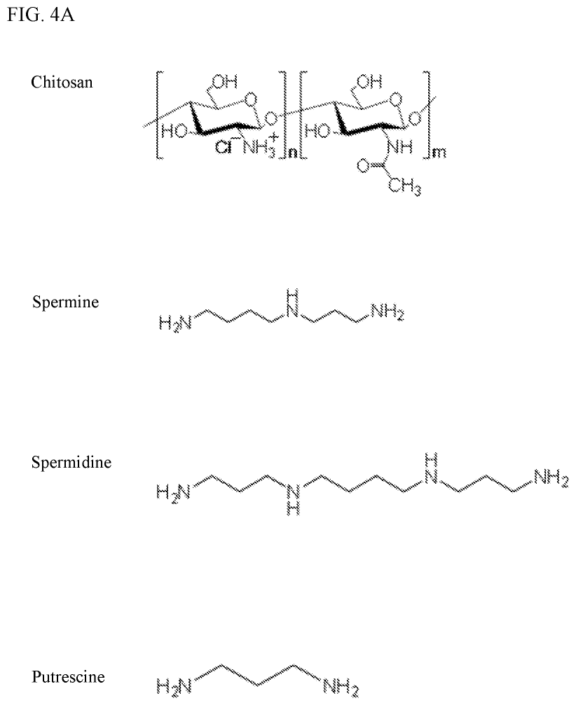

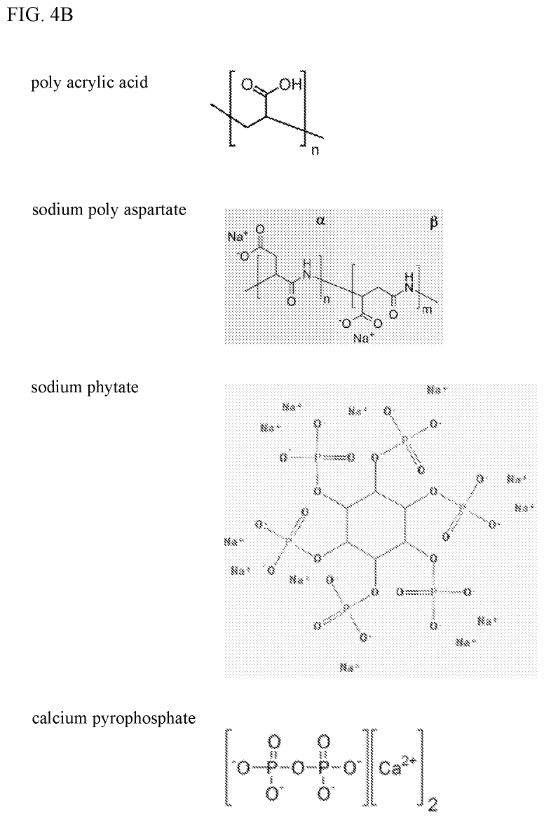

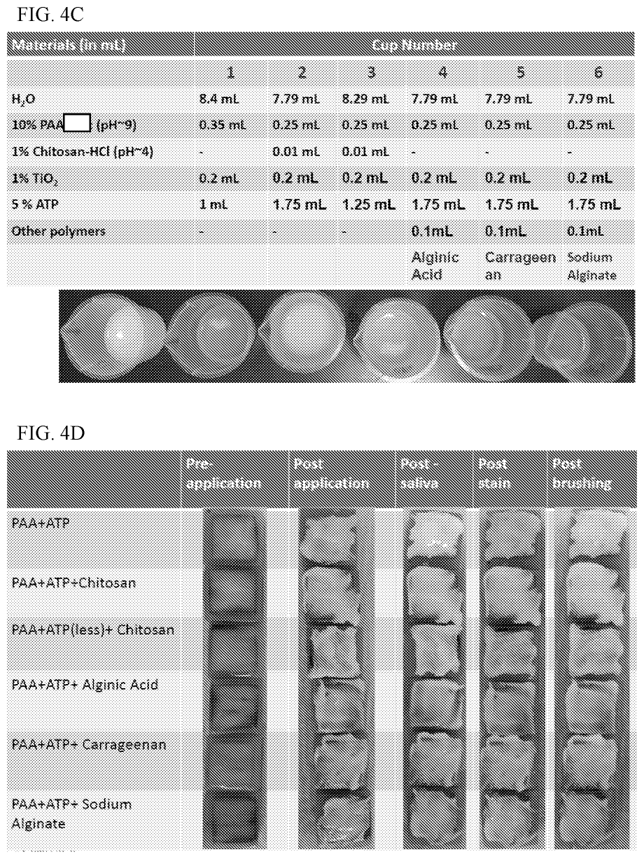

Polyelectrolyte adhesive coatings containing polyamines including amines derived from natural sources and their formulations are shown in FIG. 4C. Polyelectrolyte adhesive coatings containing polyamines including amines derived from natural sources are shown before application, after application, after exposure to saliva, after exposure to staining, and after brushing in FIG. 4D.

### Example 2: Polyelectrolyte Adhesive with Whitening Agents

Polyelectrolyte adhesives containing various whitening agents were tested for having various appearances and properties (e.g., clear, translucent, glossy, shiny, opaque, etc.) when applied to teeth.

A polyelectrolyte adhesive coating containing a whitening agent was applied to the teeth using a suitable brush. Whitening agents examined include Aluminium Oxide (50 nm), Silicon Dioxide (SiO2, 20-30 nm), Zinc Oxide (ZnO), Calcium Oxide (CaO), Amorphous Silica (SiO₂), Zirconium Carbonate, Hydroxy Apatite (HA, 20 nm, needle shaped), Functionalized TiO₂, Kronos Anatase-food grade (Standard), SiO₂ (20-30 nm) Coated with Silane KH550, ZnO (20-30 nm) Coated with Silane KH550, HA nano powder from SIGMA.

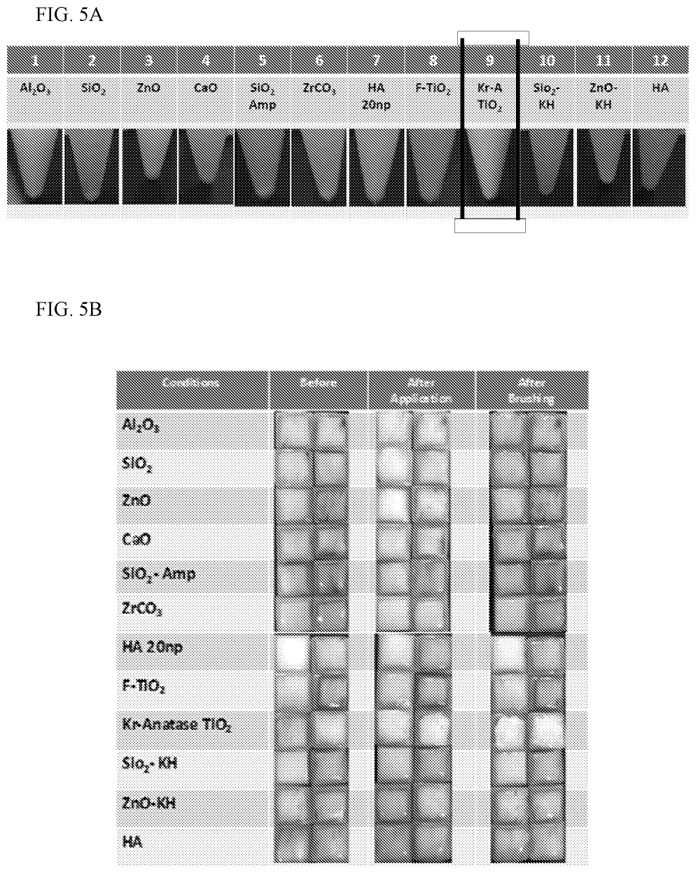

Polyelectrolyte adhesive preparations with whitening agents are shown in FIG. 5A. Polyelectrolyte adhesive preparations with whitening agents applied to teeth before and after brushing are shown in FIG. 5B. These results demonstrate that different whiteners can be used in combination with TiO₂ to develop a natural look.

### Example 3: Polyelectrolyte Adhesive with Rigidity Promoter

Polyelectrolyte adhesives containing ZnO were tested for having resistance to mechanical abrasion (e.g., toothbrushes, toothpicks, hard foods such as chips and almonds) and staining when applied to teeth.

Polyelectrolyte adhesives containing ZnO were applied to teeth. An exemplary polyelectrolyte adhesive included about 1:1 PAA:STP ratio, optimized pH , a 0.02% TiO₂, and 0.2% ZnO. The polyelectrolyte adhesive was allowed 5-30 minutes of reaction time. The teeth were incubated in artificial saliva for 2 hours, and stained in a 6% coffee solution for 10 minutes at 37° C.

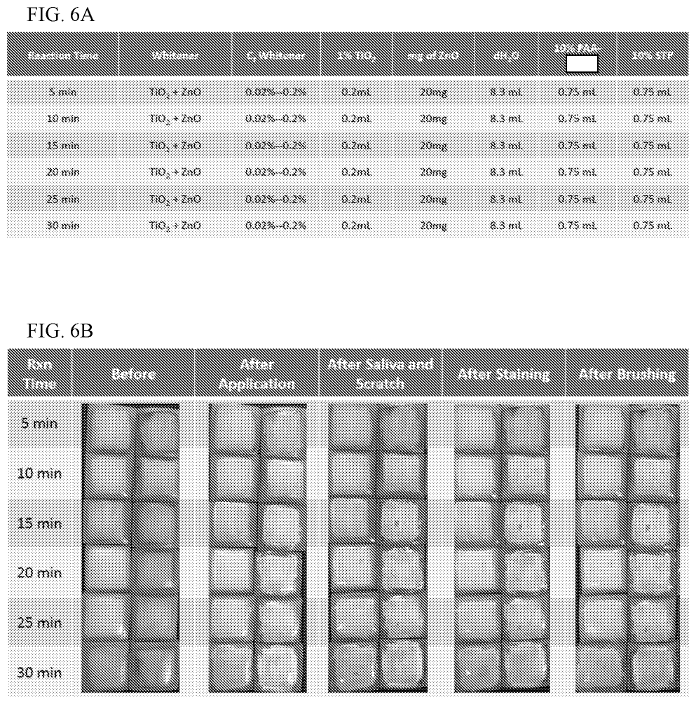

Polyelectrolyte adhesive preparations with and reaction times are shown in FIG. 6A. Polyelectrolyte adhesive coatings containing ZnO are shown before application, after application, after exposure to saliva and scratching, after exposure to staining, and after brushing in FIG. 6B.

### Example 4: Polyelectrolyte Adhesive with Flavoring Agent

Polyelectrolyte adhesives containing flavoring agents (e.g., peppermint oil) were applied to teeth. An exemplary polyelectrolyte adhesive included about at 1:6.67 PAA:STP ratio, a pH of 9, 0.02% TiO₂, and 0.1 to 5 wt % peppermint oil. The polyelectrolyte adhesive was applied with an applicator.

### Example 5: Application of Polyelectrolyte Adhesive

A uniform and easy to apply coating requires an applicator that is compatible with the polyelectrolyte adhesive coating, and allows ease of application, a uniform application with no lasting brush marks or other implement markings, and a long lasting coating.

### Applicator Brushes

Various applicator brushes were used to apply a polyelectrolyte adhesive onto the tooth surface to form a coating. The formulation reaction was strictly controlled through the correct use of pH, type of phosphates, type of amines, amine to phosphate ratio, concentration of reactants, and reaction time as described in Example 1.

Polyelectrolyte adhesive coatings applied to teeth using cosmetic angled eye liner brushes and eye shading brushes, and synthetic polyethylene brushes are shown in FIG. 7. These results demonstrate that small, dense cosmetic brush can apply a polyelectrolyte adhesive to teeth in a uniform manner.

### Applicator Strips

Applicator strips were used to apply a polyelectrolyte adhesive onto the tooth surface to form a coating.

A polyelectrolyte adhesive was prepared as shown in the below table

| Reaction Time | dH₂O | 1% TiO₂ | 10% PAA-PS | 10% STP |
|---|---|---|---|---|
| 10-30 min | 4.05 mL | 0.2 mL | 0.75 mL | 5 mL |

The preparation was adjusted to pH 9, had a PAA:STP ratio of 1:6.67, and had a PAA C_{f} of 0.75%. Three conditions (Mechanical mixing, No Mixing (Control), and No Mixing with water replaced with 1× PBS) of preparation were tested.

The polyelectrolyte adhesive was spread onto a dissolvable food grade strip and allowed to dry. The strip was then applied to the tooth surface and allowed to dissolve, transferring the adhesive to the tooth.

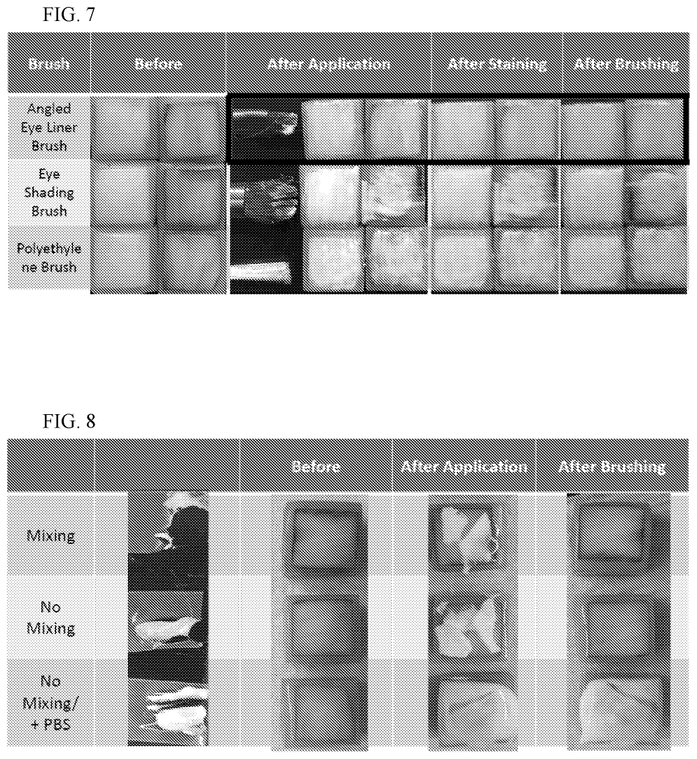

Polyelectrolyte adhesive coatings applied to teeth using a strip are shown before application, after application, and after brushing in FIG. 8.

### Application Powders

Powder polyelectrolyte adhesives were made through various drying processes (e.g., lyophilization) and were used to apply onto the tooth surface to form a coating.

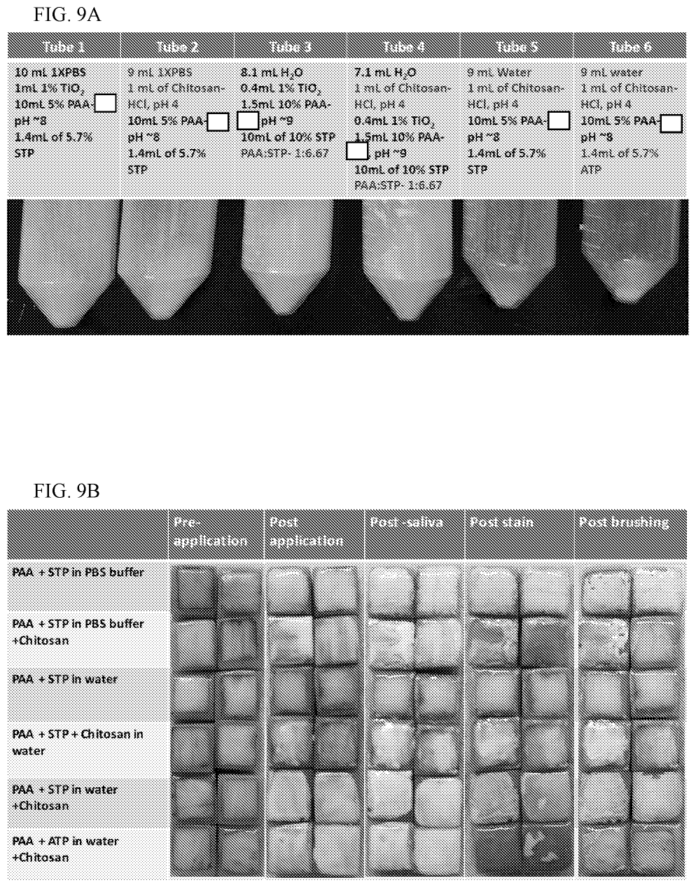

Powder polyelectrolyte adhesive preparations are shown in FIG. 9A. Powder polyelectrolyte adhesive coatings applied to teeth are shown before application, after application, after exposure to saliva, after exposure to staining, and after brushing FIG. 9B.

### Example 6: Polyelectrolyte Adhesive Post-Treatment

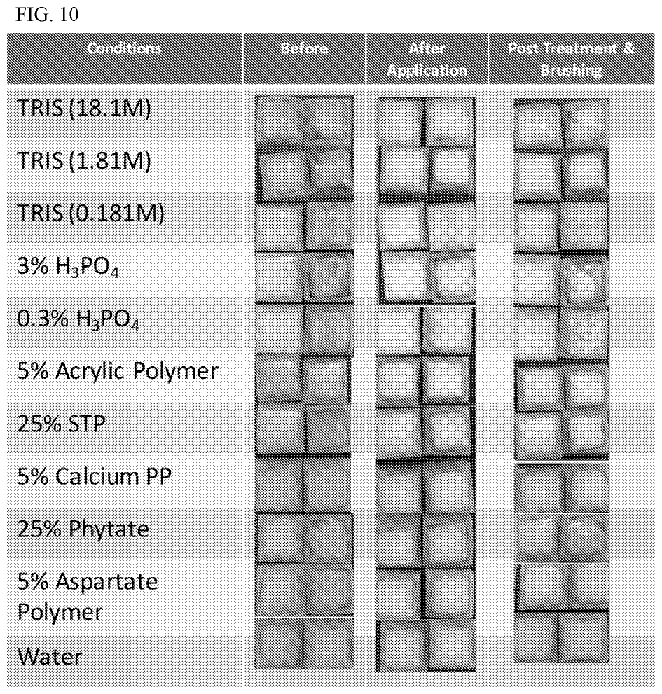

Polyelectrolyte adhesive coatings having a PAA:STP ratio of 1:6.67 were applied to teeth, teeth were brushed, and various post treatment conditions were examined. Post treatment reagents included sodium phytate, poly acrylic acid, sodium polyaspartate, TRIS base, phosphoric acid, sodium tri metaphosphate, and calcium pyrophosphate. Results of post treatments are shown in FIG. 10.

### Example 7: Polyelectrolyte Adhesive Removal

Methods for removing a polyelectrolyte adhesive coating from teeth were examined. Methods included physical abrasion using pumice (e.g., clinical pumice, coarse pumice, and fine pumice) and dental scaling, and reagents including 2-(N-morpholino)ethanesulfonic acid (MES) paste −-11.79 M (pH ˜3-4), tris(hydroxymethyl)aminomethane (TRIS) paste −18.01 M (pH ˜12-13), NaCl paste −35.95 M (pH˜7-8), sodium carbonate (Na₂CO₃) paste −28.5 M (pH ˜11-12), Dil. Na₂CO₃ −0.95 M (pH˜11-12), 3% phosphoric acid (pH˜2-3), 30% phosphoric acid (pH A-1), 0.1M HCl (pH ˜2-3), and 1M HCl (pH˜0-1).

Tooth blocks were coated with a polyelectrolyte adhesive containing a 1:1 PAA:STP ratio, optimized pH , a 0.02% to 0.5% combination of TiO₂, and other opacifiers. After coating, the tooth blocks were incubated in water for 1 hour at room temperature. Tooth blocks were removed from the water and brushed in water with spin toothbrush for 10 seconds. The tooth blocks were soaked in various reagents for 30 seconds, followed by either brushing with a hand pumicing tool in water for 30 seconds or scaling in water for 30 seconds.

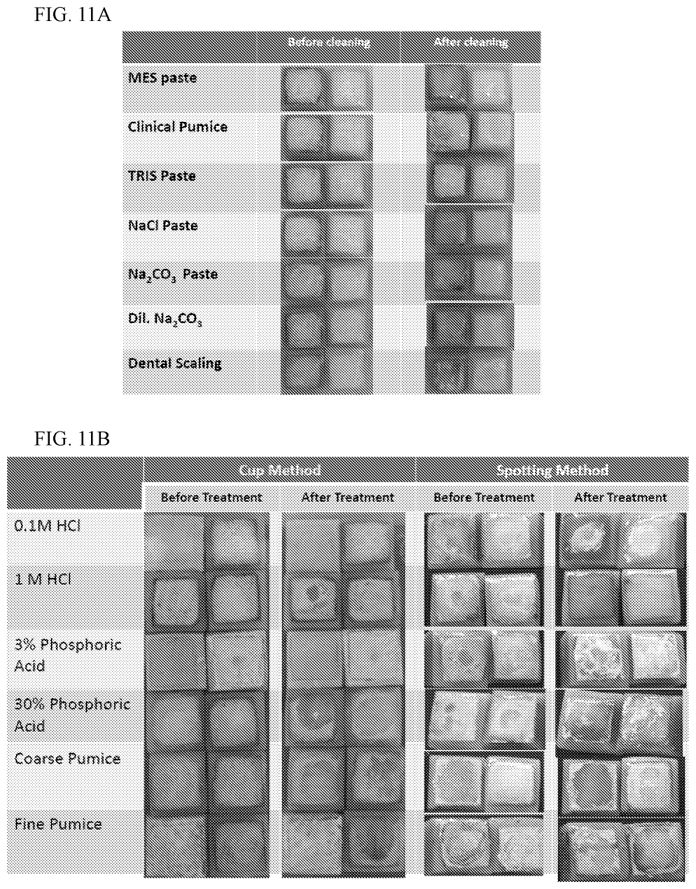

Results of exposing teeth coated with a polyelectrolyte adhesive to removal conditions are shown in FIG. 11A and FIG. 11B.

### Example 8: Clear Glossy Coating

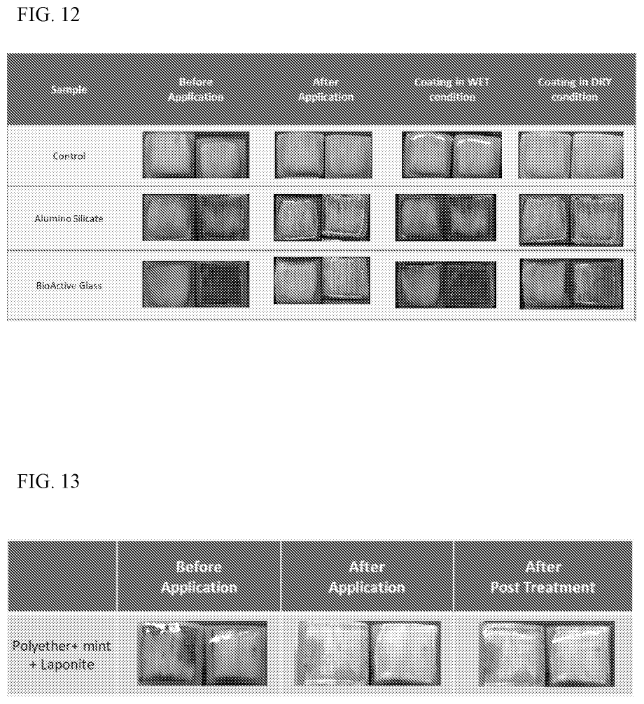

A polyelectrolyte adhesive coating, containing an opacifier was applied to the teeth using a suitable brush. Opacifiers examined include bioactive glass, alumino silicate, and mixed oxides. Bioactive glass and alumino silicate appear as a clear, glossy coat when wet and white when dry. These results demonstrate forming a clear, glossy coating on a substrate when applied and in wet conditions as shown in FIG. 12.

### Example 9: Post Treatments

Polyelectrolyte adhesive coatings having a PAA:TPP ratio of 1:1 were applied to teeth, various post treatment conditions were applied after 1 minute of drying and were examined. The post treatment reagents include polyether, laponite, and mint flavor. Results of this post treatment are shown in FIG. 13.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A polyelectrolyte adhesive comprising a conjugate of a polyamine, a polyphosphate, and a whitening agent and having the ability to interact with or bind to a substrate under wet conditions, and wherein said whitening agent of said polyelectrolyte adhesive provides said substrate with a white appearance when said polyelectrolyte adhesive is adhered to said substrate.

2. The polyelectrolyte adhesive of claim 1, wherein said substrate is selected from the group consisting of a tooth, a tooth component, an inorganic dental material, skin, hair, and nails.

3. The polyelectrolyte adhesive of claim 1, wherein said substrate comprises a tooth, and wherein said tooth is a human tooth.

4. The polyelectrolyte adhesive of claim 1, wherein said polyamine comprises a synthetic polyamine, and wherein said synthetic polyamine is selected from the group consisting of polyallylamine (PAA), polydiallylamine (DAA), polyampholite, polylysine, polyvinyl amine, polyethyleneimine (PEI), heparin, polymethacrylate, and polyhistidine.

5. The polyelectrolyte adhesive of claim 1 any one of claims 1-3, wherein said polyamine comprises a naturally-derived polyamine, and wherein said naturally-derived polyamine is selected from the group consisting of deacetylated chitosan, soy-based polyamines, spermine, spermidine, putrescine, polyamines extracted from wheat germ, and polyamines extracted from rice.

6. The polyelectrolyte adhesive of claim 1, wherein said polyphosphate is selected from the group consisting of sodium hexametaphosphate, sodium trimetaphosphate, sodium tripolyphosphate, sodium pyrophosphate, potassium tripolyphosphate, potassium pyrophosphate, and adenosine triphosphate.

7-8. (canceled)

9. The polyelectrolyte adhesive of claim 1, wherein said whitening agent comprises at least one of titanium dioxide, hydroxyapatite, silicon dioxide, zinc oxide, aluminum oxide, zirconium oxide, sodium silicate, and bioactive glass.

10-19. (canceled)

20. A method for altering an appearance of a substrate, said method comprising applying to said substrate a polyamine, a polyphosphate, and a beneficial agent, to form a polyelectrolyte adhesive on a surface of said substrate, wherein said beneficial agent of said polyelectrolyte adhesive alters said appearance of said substrate.

21. The method of claim 20, wherein said substrate is selected from the group consisting of a tooth, a tooth component, an inorganic dental material, skin, hair, and nails.

22. The method of claim 21, wherein said substrate comprises a tooth, and wherein said tooth is a human tooth.

23. The method of claim 20, wherein said beneficial agent is a whitening agent, and wherein said method comprises altering said appearance of said substrate such that said substrate appears whiter.

24. The method of claim 23, wherein said whitening agent comprises at least one of titanium dioxide, hydroxyapatite, silicon dioxide, zinc oxide, aluminum oxide, zirconium oxide, sodium silicate, alumino silicate, and zirconium silicate, and bioactive glass.

25. The method of claim 24, wherein said whitening agent comprises TiO₂, and wherein said TiO₂ comprises from about 0.005% to about 10% of said polyelectrolyte adhesive.

26. The method of claim 20, wherein said beneficial agent is an opacifier, and wherein said method comprises altering said appearance of said substrate such that said substrate appears more natural looking.

27. The method of claim 26, wherein said opacifier is selected from the group consisting of apa apatite, hydroxyapatite, mica, titanated mica, zinc oxide, zirconium oxide, aluminum oxide, boron trioxide, bioactive glass, sodium oxide, sodium silicate, alumino silicate, and zirconium silicate.

28. The method of claim 20, wherein said polyamine comprises a synthetic polyamine, and wherein said synthetic polyamine is selected from the group consisting of polyallylamine (PAA), polydiallylamine (DAA), polyampholite, polylysine, polyvinyl amine, polyethyleneimine (PEI), heparin, polymethacrylate, and polyhistidine.

29-51. (canceled)

52. A method for removing a polyelectrolyte adhesive from a substrate, wherein said polyelectrolyte adhesive comprises a polyamine, a polyphosphate, and a beneficial agent, said method comprising contacting said polyelectrolyte adhesive with a removal agent.

53. The method of claim 52, wherein said removal agent is selected from the group consisting of TRIS, phosphoric acid, 1M HC1, polyphosphate, EDTA, and NaCl.

54. The method of claim 52, wherein said removal agent is a natural removal agent.

55. The method of claim 52, wherein said substrate comprises staining, and wherein removal of said polyelectrolyte adhesive further comprises removal of staining from said substrate.

56-64. (canceled)
